(19) United States
(12) Patent Application Publication
(10) Pub. No.: US 2013/0157364 A1
(43) Pub. Date: Jun. 20, 2013

(54) MEDIUM COMPOSITION FOR CULTURING SELF-ACTIVATED LYMPHOCYTES AND METHOD FOR CULTURING SELF-ACTIVATED LYMPHOCYTES USING SAME

(71) Applicants: Seon Min Hong, Yongin-si (KR); Young Seok Baek, Bucheon-si (KR)

(72) Inventors: Seon Min Hong, Yongin-si (KR); Young Seok Baek, Bucheon-si (KR)

(73) Assignee: CELLTECH CO., LTD., Seongnam-si, Gyeonggi-do (KR)

(21) Appl. No.: 13/819,426

(22) PCT Filed: Jun. 1, 2011
(86) PCT No.: PCT/KR11/04013

§ 371 Date: Feb. 27, 2013

(30) Foreign Application Priority Data
Aug. 30, 2010 (KR) 10-2010-0083836

(51) Int. Cl. C12N 5/0783

(52) U.S. Cl. CPC C12N 5/0636
USPC 435/375

(57) ABSTRACT

Disclosed is a medium composition for culturing self-activated lymphocytes, which contains anti-CD3 antibody and anti-CD16 antibody in addition to interleukin 2 (IL-2), interleukin 12 (IL-12) and interleukin 18 (IL-18) in a medium, and thus can efficiently proliferate and activate NK cells, T cells and NKT cells and, at the same time, can significantly increase the ratio of NK cells in lymphocytes so as to provide immunocytes having excellent effects on the treatment of various kinds of malignant tumors, and a method for culturing self-activated lymphocytes using the medium composition.

### TECHNICAL FIELD

The present invention relates to a medium composition for culturing self-activated lymphocytes which are used for the treatment of malignant tumors, and a method for culturing self-activated lymphocytes using the medium composition. More specifically, the present invention relates to a medium composition for culturing self-activated lymphocytes, which contains anti-CD3 antibody and anti-CD16 antibody in addition to interleukin 2 (IL-2), interleukin 12 (IL-12) and interleukin 18 (IL-18) in a medium, and thus can efficiently proliferate and activate NK cells, T cells and NKT cells and, at the same time, can significantly increase the ratio of NK cells in lymphocytes so as to provide immunocytes having excellent effects on the treatment of various kinds of malignant tumors, and a method for culturing self-activated lymphocytes using the medium composition.

### BACKGROUND ART

Recently, adaptive immunotherapy has drawn attention as new, alternative cancer treatment method to surgery, radiation therapy or chemotherapy conventionally used in the treatment of cancers.

Adaptive immunotherapy is a method involving extracting natural killer (NK) cells, dendritic (DC) cells, B cells, T cells, and the like, which are the most crucial immunocytes for the treatment of cancers, from blood of a patient, incubating the extracted immunocytes with different kinds of stimulants so as to strongly act against cancer cells, and then injecting them back into the patient. Since the patient's own blood is used, the adaptive immunotherapy causes less side effects and is performed using a more convenient administration method than conventional chemotherapies and the like. For these reasons, adaptive immunotherapy is currently being actively researched.

NK cells among immunocytes which are activated in adaptive immunotherapy are a kind of lymphocytes that have an excellent ability to kill infected viruses and tumor cells, but not to kill most normal cells. Such. NK cells are known to play an important role in the initial defensive mechanism and the human tumor immunity. In other words, NK cells can kill specific cancer cells, homologous cells and even heterologous cancer cells without the acquisition of immunity resulting from the expression of major histocompatibility complex (MHC). In particular, NK cells can more effectively kill target cells which express little or no Class 1 MHC. Thus, NK cells can effectively kill most cancer cells that do not express MHC, cells infected with several viruses, and bacteria such as Salmonella typhi.

However, NK cells, which have a superior ability to kill cancer cells, account for only 5-15% of peripheral blood lymphocytes even in normal people, and their proportion is reduced to 1% or less in cancer patients. Thus, NK cells cannot effectively attack cancer cells, when they are not subjected to a separate proliferation process in adaptive immunotherapy.

Thus, to apply adaptive immunotherapy to cancer therapy, it is essential to conduct studies on a medium for culturing and activating large amounts of immunocytes, including NK cells, on a large scale according to the progression stage of the patient's cancer. However, conventional immunocyte culture media used in adaptive immunotherapy have been focused mainly on proliferating T cells among immunocytes on a large scale.

A conventional medium for culturing immunocytes is disclosed in Korean Patent Registration No. 0735081, entitled “A method for activating CD4 T cells”. The method for activating CD4 T cells involves isolating CD4 T cells from a biological sample such as blood and culturing the isolated CE4 T cells in a medium containing cytokines, including GM-CSF, IFN-gamma, TNF-alpha, lectin and IL-4, to activate the CD4 T cells in vitro. As a result, a composition for preventing or treating bacterial infectious diseases can be obtained.

However, in the medium composition that is used in the method for activating CD4 T cells, only T cells among immunocytes, which are involved in acquired immunity, are selectively activated. Thus, when the activated. T cells are used for tumor treatment, they can effectively attack and kill cancer cells that they memorize. However, it is difficult for the activated T cells to treat malignant tumors by attacking various kinds of cancer cells that they cannot memorize.

In an attempt to overcome this problem, Korean Patent Laid-Open Publication No. 2008-0053929 (entitled “Method for culturing self-activated lymphocytes) filed in the name of the applicant discloses a method in which lymphocytes isolated from human peripheral blood are cultured in the presence of interleukin 2 (IL-2), anti-CD3, anti-CD16 and anti-CD56 antibodies to increase the ratio of NK cells in the lymphocytes, so that the activated NK cells, T cells and NKT cells can be uniformly distributed to effectively remove various kinds of cancer cells.

FIGS. 1 and 2 are graphs showing changes in the phenotype of activated lymphocytes obtained by the above method for culturing self-activated lymphocytes. In FIGS. 1 and 2, the H1 region represents the distribution of NK cells, the H4 represents the distribution of T cells, H2 region represents the distribution of NKT cells, and the H3 region represents the distribution of other immunocytes.

The surface antigens of the activated lymphocytes obtained by the above method for culturing self-activated lymphocytes were analyzed by flow cytometry. As a result, as can be seen in FIGS. 1 and 2, the surface antigens were most densely distributed in the H4 region before culture as shown in FIG. 1, but were most densely distributed in the H1 region after culture as shown in FIG. 2.

The ratio of CD16+CD56 positive NK cells in the lymphocytes cultured as described above was increased from 12.74% before culture to 63.42% after culture.

The cytotoxicity of the activated lymphocytes cultured as described above was analyzed to determine the ability to kill blood cancer cells. As a result, it could be seen that the cytotoxicity of the activated lymphocytes was 6-32 times higher than that of lymphocytes extracted from general blood.

As described above, it can be seen that, when immunocytes are activated, the ratio of NK cells therein and the cytotoxicity thereof can be increased so that their ability to kill cancer cells can be increased, suggesting that they have increased anticancer effects.

Accordingly, there have been extensive studies on medium compositions for culturing activated lymphocytes, in which the medium compositions can efficiently proliferate and activate large amounts of NK cells among lymphocytes, which have an excellent ability to kill most cancer cells which do not express MHC, and the medium composition can increase the ability of NK cells to kill cancer cells so that the NK cells have increased anticancer activity, and methods for culturing activated lymphocytes.

### DISCLOSURE

### Technical Problem

Accordingly, the present invention has been made keeping in mind the above problems occurring in the prior art, and an object of the present invention is to provide a medium composition for culturing self-activated lymphocytes, which contains anti-CD3 antibody and anti-CD16 antibody in addition to interleukin 2 (IL-2), interleukin 12 (IL-12) and interleukin 18 (IL-18) in a medium, and thus can efficiently proliferate and activate NK cells, T cells and NKT cells and, at the same time, can significantly increase the ratio of NK cells in lymphocytes so as to provide immunocytes having excellent effects on the treatment of various kinds of malignant tumors, and a method for culturing self-activated lymphocytes using the medium composition.

### Technical Solution

In order to accomplish the above object, the present invention provides a medium composition for culturing self-activated lymphocytes, the medium composition comprising: a cell culture medium; and additives that are added to the cell culture medium, wherein the additives include interleukin 2 (IL-2), interleukin 12 (IL-12), interleukin 18 (IL-18), anti-CD3 antibody and anti-CD16 antibody.

The present invention also provides a method for culturing self-activated lymphocytes the steps of: extracting lymphocytes from human peripheral blood; culturing the extracted lymphocytes in a medium containing interleukin 2 (IL-2), L-glutamine and autologous plasma in the presence of anti-CD3 and anti-CD16 antibodies (first culture step); and 3) adding the lymphocyte-containing medium resulting from the first culture step to a medium containing IL-2, L-glutamine and autologous plasma, followed by culture in the presence of IL-12, anti-CD16 antibody and IL-18 (second culture step).

### Advantageous Effects

A medium composition for culturing self-activated lymphocytes according to the present invention and a method for culturing self-activated lymphocytes using the same can efficiently proliferate and activate NK cells, T cells and NKT cells and, at the same time, can increase the ratio of NK cells in the lymphocytes, so that the activated NK cells can kill most cancer cells which do not express MHC. Thus, the medium composition and method of the present invention can be applied to adoptive immunotherapy, which has less side effects and uses a convenient administration method, thereby greatly improving the prognosis of cancer patients suffering from various kinds of malignant tumors.

### DESCRIPTION OF DRAWINGS

FIG. 1 is a graph showing a change in the phenotype of activated lymphocytes obtained by a culture method according to the prior art.

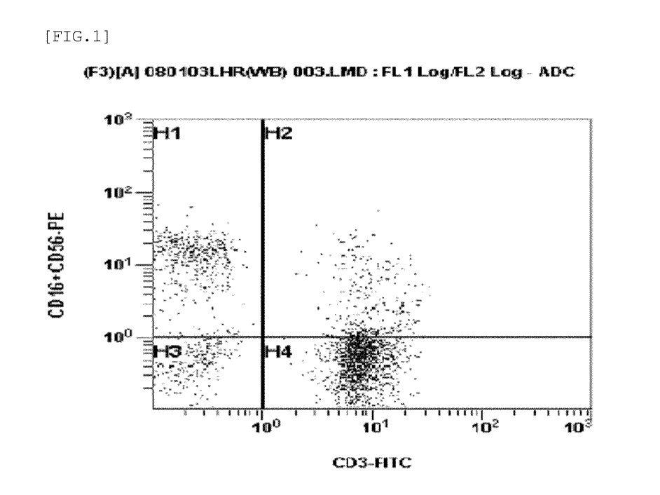

FIG. 2 is a graph showing a change in the phenotype of activated lymphocytes obtained by a culture method according to the prior art.

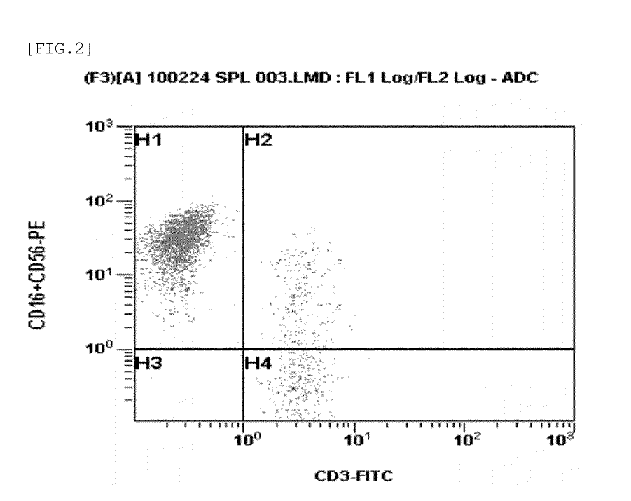

FIG. 3 is a graph showing a change in the phenotype of activated lymphocytes obtained by a method for culturing self-activated lymphocytes according to the present invention.

FIG. 4 is a graph showing a change in the phenotype of activated lymphocytes obtained by a method for culturing self-activated lymphocytes according to the present invention.

FIG. 5 is a graph showing a comparison between a change in the number of NK cells obtained by a method for culturing self-activated lymphocytes according to the present invention and a change in the number of NK cells obtained by a method for culturing self-activated lymphocytes according to the prior art.

FIG. 6 is a graph showing a comparison of cytotoxicity between activated lymphocytes obtained by a method for culturing self-activated lymphocytes according to the present invention and activated lymphocytes obtained by a method for culturing self-activated lymphocytes according to the prior art.

### BEST MODE

The present invention is directed to a method for producing self-activated lymphocytes having excellent effects on the treatment of malignant tumors, wherein lymphocytes derived from human peripheral blood are cultured in a medium containing interleukin 12 (IL-12), interleukin 18 (IL-18), anti-CD3 antibody and anti-CD16 antibody as additives to efficiently proliferate and activate NK cells, T cells and NKT cells while increasing the ratio of NK cells in the lymphocytes.

Before describing the present invention in detail, the characteristics of each kind of immunocytes and the mechanism of action of the additives that are used in the proliferation and activation of immunocytes will be explained.

NK cells are large granular lymphocytes (LGLs), a kind of lymphocytes, and show antitumor activity via necrosis, apoptosis or a combination thereof. NK cells respond to cytokines such as IL-2, IL-12 and interferon, so that the cytotoxicity, secretory and proliferative activities thereof increase. The phenotypes of NK cells are CD16 (FcγRIII) and CD56, and CD16 and CD56 have no TRC (T-cell receptor complex) on the cell surface, and thus are used as NK cell markers.

IL-2 is a glycoprotein having a molecular weight of 14-17 kDa, which is produced when T cells are activated by recognition of antigen. After IL-2 has been secreted out of T cells, it reacts with the T cells that produced IL-2, thus promoting the growth of the corresponding T cells. IL-2 also acts on NK cells to promote the growth of the cells and enhance the cytotoxicity of NK cells. In addition, it acts on B cells to promote the growth of the cells.

IL-12 is produced in dendritic cells (DCs), macrophages and B cells. IL-12 induces the production of IFN-γ and TNF-α in NK cells and T lymphocytes and functions to reduce the production of IL-4 that suppresses IFN-γ. It also increases the cytotoxicity of NK cells and CD8+ cytotoxic T lymphocytes. IL-12 has a close connection with the IL-2 signaling system in NK cells. In addition, IL-2 induces the expression of IL-12 receptor 131 and IL-receptor 132 in NK cells to express and activate proteins related to the IL-12 signaling system. This mechanism was well demonstrated from the abilities of NK cells to produce IFN-γ and kill target cells. The IL-12 receptor 132 appears to play an important role in the IL-12 function and was reported to inhibit the generation of Th2 while inducing the generation of Th1. IL-12 signaling in T cells and NK cells is involved in the JAK-STAT signaling system, and the activity of the IL-12 receptor 132 plays an important role in inducing the phosphorylation of the transcription factor STAT4 to activate the STAT4.

IL-18 is a proinflammatory cytokine gene that is produced in macrophages. IL-18 binds to the IL-18 receptor to induce an immune response in virus- or bacteria-infected cells together with IL-12. In addition, it produces the production of IFN-γ in NK cells and T cells. In the present invention, IL-12 is added to the medium to induce the expression of INF-γ while inducing the expression of the IL-18 receptor essential for the production of INF-γ to thereby activate NK cells.

The mechanism of action of NK cells on cancer cells is generally antibody-dependent cell mediated cytotoxicity (ADCC). NK cells express CD16, a receptor for Fc of immunoglobulin G (IgG), and can perform MHC-unrestricted killing by the receptor. In other words, the ADCC of NK cells depend on the presence of an antibody that recognizes target cells, and when the antibody binds to an antigen, the Fc portion of the antibody is exposed. When the exposed Fc portion binds to the receptor of NK cells to form a bridge, a cytotoxic substance is released from the NK cells by signaling mediated by the receptor and damages target cells.

When lymphocytes are cultured in the presence of anti-CD16 antibody or an antigen-antibody complex, the CD16 antigen is added to NK cells to cause signaling. Due to the stimulation of the NK cells with the antibody and the antigen, a transferrin receptor such as the α chain of the IL-2 receptor can be expressed in the NK cells, or tumor necrosis factor (TNF) or IFN-γ can be produced in the NK cells.

Meanwhile, T cells refer to cells having T cell receptor (TCR) on the cell surface. TCR forms a heterodimer with CD3 antigen, a dimer of α- and β-chains. Some of T cells (about 5% of peripheral blood T cells) are comprised of a dimer of γ- and δ chains other than αβ chains. TCR forms a complex with CD3 antigen (γ, δ, ε, ζ, ζor η), and when the CD3 antigen is recognized by TCR, it transmits the signal into cells.

Generally, helper T cells that stimulate an immune response to cancer cells release various cytokines to activate killer T cells, B cells, macrophages, NK cells and the like. Examples of the cytokines include IL-1, IL-2, IL-3, TNF-α and IFN-γ, which are cell-to-cell messengers. In the present invention, a high concentration of IL-2 is added to the medium to activate immunocytes, including T cells, NK cells and NKT cells.

NKT cells are a kind of T cells, which involve innate immunity and whose functions have recently been found. As can be seen from their name, NKT cells express T cell receptor and NK cell specific surface markers. One surprising characteristic of NKT cells is that they secrete various cytokines such as IL-4, IL-10, IL-13, IFN-γ and TNF-α within a very short time after activation. This characteristic suggests that NKT cells may have a great influence on adaptive immunity.

Thus, in the present invention, in order to activate NK cells, T cells and NKT cells among lymphocytes from human peripheral blood, IL-2, IL-12 and IL-18 cytokines and anti-CD3 and anti-CD16 monoclonal antibodies are used as medium additives during culture. Among the additives, IL-2, IL-12 and IL-18 function to stimulate the proliferation of T cells and NK cells, and the monoclonal antibodies function as antigens that express CD3 and CD16 in immunocytes.

Hereinafter, the method for culturing self-activated lymphocytes according to the present invention will be described in detail.

The method for culturing self-activated lymphocytes according to the present invention generally comprises a lymphocyte extraction step, a first culture step, a second culture step and a third culture step.

A medium composition which is used in each of the culture steps is as follows.

First Medium Composition

A first medium composition is used in the first culture step and comprises a cell culture medium, and various additives added to the cell culture medium. Specifically, the first medium composition comprises, based on 39 ml of the cell culture medium, 350-430 μl of 190-210 mM of L-glutamine and 2-5 ml of autologous plasma, which serve as nutrients for culture of immunocytes, 3-10 μl of 17×10⁶−19−10⁶ IU/ml of IL-2, 3-10 μl of 90-110 μg/ml of IL-12 and 10-30 μl of 90-110 μg/ml of IL-18, which serve to activate immunocytes, and 2-30 μl of 0.9-1.1 mg/ml of each of anti-CD3 and anti-CD16 antibodies, which serve to control the ratio of the cultured immunocytes (NK cells, NKT cells and T cells).

When lymphocytes are cultured using this medium composition, a subculture method is preferably used in order to increase culture efficiency. Specifically, lymphocytes are cultured using the above medium composition for 3-4 days, and then the medium composition having the lymphocytes cultured therein is incorporated into the following second medium composition, followed by additional culture for 1-2 days.

Second Medium Composition

The second medium composition is used for subculture and comprises a cell culture medium, and additives added to the cell culture medium. Specifically, the second medium composition comprises, based on 67 ml of the cell culture medium, 650-740 μl of 190-210 mM L-glutamine and 2-5 ml of autologous plasma, which serve as nutrients for culture of immunocytes, 3.5-7.8 μl of 17×10⁶−19×10⁶ IU/ml IL-2, 2-10 it of 90-110 μg/ml and 20-50 μl of 90-110 μg/ml IL-18, which serve to activate immunocytes, and 4-80 μl of 0.9-1.1 mg/ml anti-CD16 antibody which serves to control the ratio of the cultured immunocytes (NK cells, NKT cells and T cells).

The cell culture medium that is used in each of the first and second medium compositions is a conventional medium containing nutrients essential for the growth and survival of cells, including amino acids, vitamins, organic and inorganic compounds, and proteins, as well as 800-1200 IU (International Unit)/ml of IL-2. For example, it may be NKB6040 (NKBIO, KOREA).

Hereinafter, the inventive method of culturing self-activated lymphocytes using the above-described medium compositions will be described in detail.

The method for culturing self-activated lymphocytes according to the present invention generally comprises a lymphocyte extraction step, a first culture step, a second culture step and a third culture step.

Lymphocyte Extraction Step

Human mononuclear cells such as lymphocytes or monocytes have a specific gravity lower than 1.077. Based on this property, the blood of the patient to be treated is layered onto the Ficoll-Paque Plus solution having a specific gravity of 1.077 and precipitated by centrifugation to separate the blood into a lower layer comprising erythrocytes and granulocytes having a specific gravity higher than 1.077 and an upper layer comprising mononuclear cells (including lymphocytes) and platelets having a specific gravity lower than 1.077, and lymphocytes are extracted from the separated mononuclear cell layer.

First Culture Step

The lymphocytes harvested by the lymphocyte extraction step are cultured in a medium containing IL-2, L-glutamine and autologous plasma in the presence of IL-12, IL-18, anti-CD3 antibody and anti-CD16 antibody for 3-4 days. Preferably, the lymphocytes are cultured in a medium containing IL-2, L-glutamine and autologous plasma in the presence of anti-CD3 antibody, and then cultured in the presence of IL-12, anti-CD16 antibody and IL-18. A specific process for culturing the lymphocytes is as follows.

First, the harvested lymphocytes are suspended in 5 ml of a medium, and the suspension is placed in a 25-cm² first culture container, which has anti-CD3 antibody solidified therein and contains 29-32 ml of a medium supplemented with 2-10 μl of a dilution of 17×10⁶−19×10⁶ IU/ml of IL-2, 350-430 μl of 190-210 mM of L-glutamine and 2-5 ml of autologous plasma. Then, the cells are cultured in a CO₂ incubator at 37° C. for 30 minutes to 2 hours. As described above, when the lymphocytes are suspended in a small amount of a medium and then cultured in a medium supplemented with IL-2, L-glutamine and autologous serum, the loss of the lymphocytes can be reduced while the loss of IL-2, L-glutamine and autologous plasma can be prevented.

Herein, the lymphocytes are cultured in a state in which the monoclonal anti-CD3 antibody was solidified in the culture container, so that the activation of T cells is induced by anti-CD3 antibody in the initial stage of culture to induce the expression and secretion of factors that proliferate NK cells. If the lymphocytes are cultured for an excessively long time in the presence of anti-CD3 antibody, most of the cells will proliferate into T cells. For this reason, it is important to culture the cells for a suitable time in order to increase the ratio of NK cells.

Then, the culture medium in the first culture container having anti-CD3 antibody solidified therein is transferred into a 25-cm² second culture container having anti-CD3 antibody solidified therein, and it is cultured in a CO₂ incubator at 37° C. for 1-3 hours in the presence of 3-10 μl of 90-110 μg/ml of IL-12 and 2-30 μl of 0.9-1.1 mg/ml of anti-CD16 antibody, after which 10-30 μl of 90-110 μg/ml of IL-18 is added to the 25-cm² second culture container, followed by culture for 48-78 hours.

Herein, each of the first and second culture containers has 2-30 μl of 0.9-1.1 mg/ml of anti-CD3 antibody solidified therein.

As described above, IL-12 and anti-CD16 antibody are simultaneously to the second culture container. In this case, the activities of transcription factors can be increased to induce the expression of INF-γ to thereby promote the proliferation and activation of NK cells, compared to when IL-12 and anti-CD16 antibody are separately added. In addition, IL-18 is a factor essential for the production of INF-γ, and the expression of the IL-18 receptor is induced by IL-12. For this reason, IL-18 is preferably added to the second culture container after addition of IL-12.

Then, 5 ml of a medium containing IL-2, L-glutamine and autologous plasma is added to the second culture container, followed by culture for 12-18 hours. Then, the cell-containing culture medium in the second culture container is transferred into a 75-cm² third culture container, and 30 ml of a medium containing IL-2, L-glutamine and autologous plasma is added thereto, followed by culture for 2-3 days.

Second Culture Step

After completion of the first culture step, the cell-containing culture medium is added to a medium supplemented with IL-2, L-glutamine and autologous plasma and cultured in the presence of IL-12 and anti-CD16 antibody, after which IL-18 is added, followed by culture. A specific process for carrying the second culture step is as follows.

First, the cell-containing culture medium resulting from the first culture step is transferred into a 150-cm² fifth culture container containing 2-7 ml of a medium supplemented with 3.5-7.8 μl of a dilution of 17×10⁶−19×10⁶ IU/ml of IL-2, 650-740 μl of 190-210 mM of L-glutamine and 2-5 ml of autologous plasma, and 20 ml of a medium containing IL-2, L-glutamine and autologous plasma is added thereto while 2-10 μl of 90-110 μg/ml of IL-12 and 2-40 μl of 0.9-1.1 mg/ml of anti-CD16 antibody are added thereto, followed by culture for 1-3 hours. In the same manner as the first culture step, IL-12 and anti-CD16 antibody are simultaneously added so that the activities of transcription factors are increased to induce the expression of INF-γ to thereby promote the proliferation and activation of NK cells, after which IL-18 is added so that the expression of the IL-18 receptor is induced by IL-12.

Next, 20-50 μl of 90-110 μg/ml of IL-18 is added to the fourth culture container, followed by culture for 12-18 hours. Then, 40 ml of a medium containing IL-2, L-glutamine and autologous plasma is added to the fourth culture container while 2-40 μl of 0.9-1.1 mg/ml of anti-CD16 antibody is added, followed by additional culture for 12-18 hours.

Third Culture Step

After completion of the second culture step, 5-10 ml of autologous plasma is added to 86-119 ml of the cell-containing culture medium, and the cell-containing culture medium is injected into a 1-l gas permeable culture bag, followed by culture for 6-9 days, thereby proliferating the cells in large amounts.

In the same manner as the above two steps, the culture medium that is used in the third culture step may contain nutrients essential for the growth and survival of cells, including amino acids, vitamins, organic and inorganic compounds, and proteins. Preferably, it contains 100-200 IU/ml of IL-2.

Through the first, second and third culture steps as described above, the number of NK cells, T cells and NKT cells increases and the size of each kind of cells also increases. In the present invention, it could be seen that the total cell number of lymphocytes isolated from 60 cc blood was increased from 2.0×10⁶−4.0×10⁷ before culture to 1.0×10⁹−3.0×10⁹ after culture.

Hereinafter, the culture of self-activated lymphocytes using the medium composition of the present invention, changes in the phenotype and cell number of lymphocytes in the cultured self-activated lymphocytes, and the cytotoxicity of the cultured self-activated lymphocytes will be described in detail with reference to examples. It is to be understood, however, that these examples are for illustrative purposes only and are not intended to limit the scope of the present invention.

First, a process of culturing self-activated lymphocytes using the inventive medium composition for culturing self-activated lymphocytes will be described with reference to Example 1.

### Example 1

### Preparation of Self-Activated Lymphocytes

1×10⁷ lymphocytes harvested from 60 cc of the patient's peripheral blood was suspended in 1 ml of a medium, and the suspension was added to a 9 ml of a medium containing IL-2, L-glutamine and autologous plasma, after which the cells were cultured in the presence of IL-12, IL-18, anti-CD3 antibody and anti-CD16 antibody for 4 days (first culture step).

The medium used in the first culture step comprises, based on 39 ml of the medium, 400 μl of 200 mM of L-glutamine and 4 ml of autologous plasma, which serve as nutrients for culture of immunocytes, 4 μl of 18×10⁶ IU/ml of IL-2, 4 μl of 100 μg/ml of IL-12 and 20 μl of 100 μg/ml of IL-18, which serve to activate immunocytes, and 2.5 μl of 1 mg/ml of each of anti-CD3 and anti-CD16 antibodies, which serve to control the ratio of the cultured immunocytes.

Next, the cell-containing culture medium resulting from the first culture step was added to 30 ml of a medium containing IL-2, L-glutamine and autologous serum, after which the cells were additionally cultured in the presence of IL-12, IL-18 and anti-CD16 antibody for 2 hours (second culture step).

The medium used in the second culture step comprises, based on 67 ml of the medium, 670 μl of 200 mM of L-glutamine and autologous plasma, which serve as nutrients for culture of lymphocytes, 6.5 μl of 18×10⁶ IU/ml of IL-2, 8 μl of 100 μg/ml of IL-12 and 40 μl of 100 μg/ml of IL-18, which serve to activate lymphocytes, and 5 μl of 1 mg/ml of anti-CD5 antibody which serves to control the ratio of the cultured lymphocytes.

Next, 10 ml of autologous plasma was added to the cell-containing culture medium resulting from the second culture step, and the medium was injected into a gas permeable culture bag containing 1 l of a medium, after which the cells were further cultured for 7 days (third culture step).

The changes in the phenotype and cell number of the activated lymphocytes before and after culture, and the results of analysis of cytotoxicities of the lymphocytes against various cancer cells will be explained with reference to Examples 2 and 3.

### Example 2

### Observation of Change in Cell Number Before and After Culture

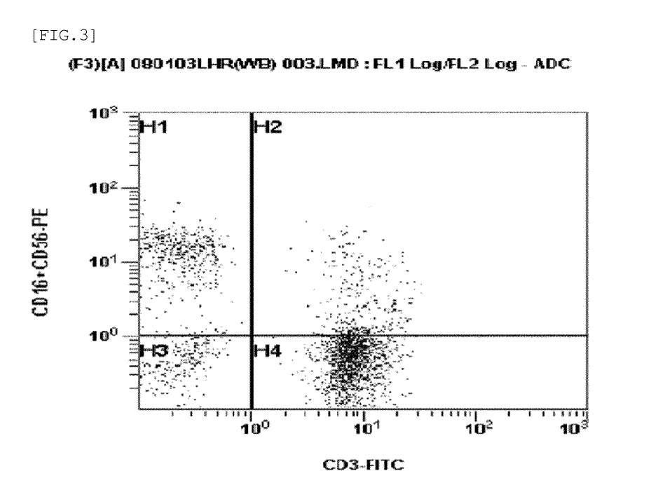

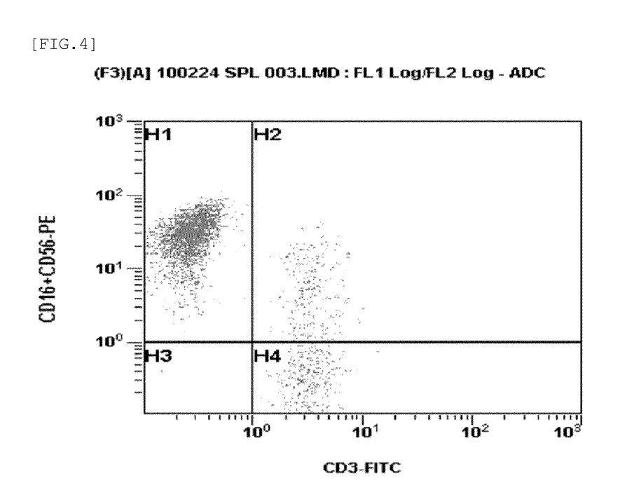

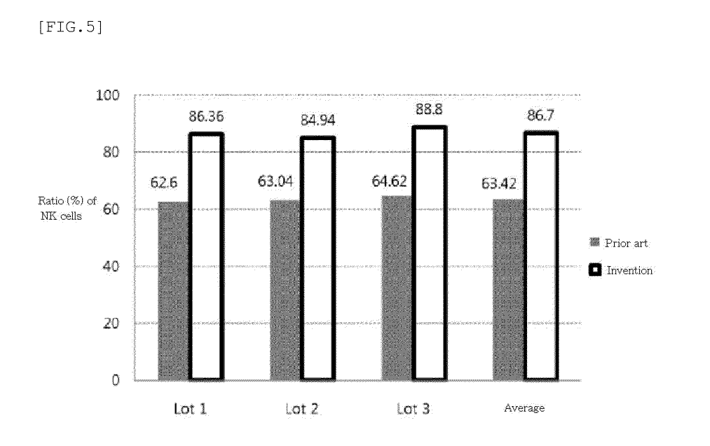

FIGS. 3 and 4 are graphs showing the change in phenotype of lymphocytes before and after culture. In FIGS. 3 and 4, the H1 region indicates the distribution of NK cells, the H4 region indicates the distribution of T cells, the H2 region indicates the distribution of NKT cells, and the H3 region indicates the distribution of other immunocytes. FIG. 5 is a graph showing a comparison between a change in the number of NK cells obtained by the inventive method for culturing lymphocytes and a change in the number of NK cells obtained by the prior art method for culturing lymphocytes.

The surface antigens of the activated lymphocytes cultured by the method of Example 1 were analyzed by flow cytometry. As a result, as can be seen in FIGS. 3 and 4, the surface antigens were most densely distributed in the H4 region before culture as shown in FIG. 3, but were most densely distributed in the H1 region after culture as shown in FIG. 4.

In addition, the ratio of CD16+CD56 positive NK cells in the lymphocytes was calculated. As a result, as can be seen in

FIG. 5, the ratio of CD16+CD56 positive NK cells in the cultured activated lymphocytes was as high as 86.7% on average.

In other words, due to the action of the additives, such as IL-12 and IL-18, added in order to activate NK cells, the ratio of NK cells in the activated lymphocytes cultured according to the present invention was about 20-30% higher than the ratio of NK cells (63.42%) in activated lymphocytes obtained by culturing lymphocytes (extracted from human peripheral blood) in the presence of IL-2 and anti-CD3, anti-CD16 and anti-CD56 antibodies according to the prior culture method (Korean Patent Laid-Open Publication No. 2008-0053929) filed in the name of the applicant. Through the analysis of cytotoxicity as described above, it could seen that this increase in the ratio of NK cells led to improved therapeutic effects.

### Example 3

### Analysis of Cytotoxicity Against Various Cancer Cells

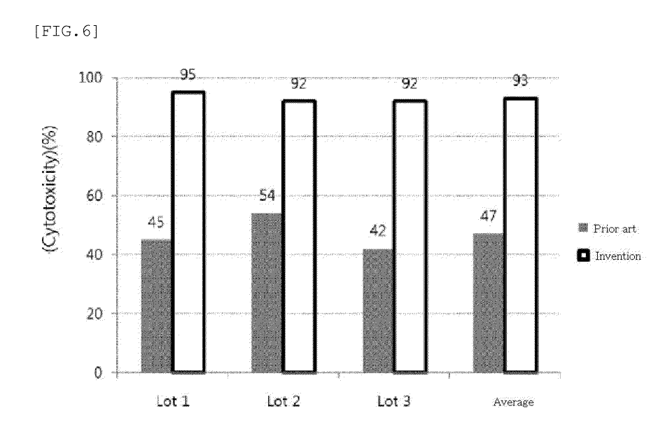

FIG. 6 shows a comparison between the cytotoxicity of the activated lymphocytes cultured by the inventive method for culturing self-activated lymphocytes and the cytotoxicity of the activated lymphocytes cultured by the prior art method for culturing self-activated lymphocytes.

In the analysis of cytotoxicity, the activated lymphocytes cultured by the method of Example 1 were used as effector cells, blood cancer cells (K562) were used as target cells. The ratio of the lymphocytes to the cancer cells was set at 10:1, and the cytotoxicity of the lymphocytes was determined by measuring the ability of the lymphocytes to kill the blood cancer cells.

In addition, under the same conditions as used in the above cytotoxicity analysis, the analysis of cytotoxicity was performed using the activated lymphocytes, cultured by the prior art culture method, as effector cells. The results of the cytotoxicity analysis are shown in FIG. 6.

As can be seen in FIG. 6, the average cytotoxicity of the activated lymphocytes cultured by the prior art method was 47%, whereas the average cytotoxicity of the activated lymphocytes cultured by the inventive method was 93%, suggesting that the cancer cell killing ability of the activated lymphocytes cultured by the invention method was about 2 times higher than that of the activated lymphocytes cultured by the prior art method. Further, because the lymphocytes used in the Examples had a cytotoxicity of 5% or less on average before culture, it can be seen that the cytotoxicity of the activated lymphocytes cultured by the present invention was at least 20 times higher than that before culture.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, the medium composition for culturing self-activated lymphocytes according to the present invention and the method for culturing self-activated lymphocytes using the same can efficiently proliferate and activate NK cells, T cells and NKT cells and, at the same time, can increase the ratio of NK cells in the lymphocytes, so that the activated NK cells can kill most cancer cells which do not express MHC. Thus, the medium composition and method of the present invention can be applied to adoptive immunotherapy, which has less side effects and uses a convenient administration method, thereby greatly improving the prognosis of cancer patients suffering from various kinds of malignant tumors.

1. A medium composition for culturing self-activated lymphocytes on a large scale, the medium composition comprising:
a cell culture medium; and additives that are added to the cell culture medium, wherein the additives include interleukin-2 (IL-2), interleukin 12 (IL-12), interleukin 18 (IL-18), anti-CD3 antibody and anti-CD16 antibody.

2. The medium composition of claim 1, wherein the cell culture medium contains 800-1,200 IU (International Unit)/ml.

3. The medium composition of claim 1, wherein the additives that are added to the cell culture medium include, based on 39 ml of the cell culture medium, 3-10 μl of 17×10⁶−19×10⁶ IU/ml of IL-2, 3-10 μl of 90-110 μg/ml of IL-12, 10-30 μl of 90-110 μg/ml of IL-18, 2-40 μl of 0.9-1.1 mg/ml of anti-CD3 antibody, and 2-40 μl of 0.9-1.1 mg/ml of anti-CD16 antibody.

4. The medium composition of claim 3, wherein the additives further include, based on 39 ml of the cell culture medium, 350-430 μl of 190-210 mM of L-glutamine and 2-5 ml of autologous plasma.

5. The medium composition of claim 1, wherein the additives that are added to the cell culture medium include, based on 67 ml of the cell culture medium, 3.5-7.8 μl of 17×10⁶−19×10⁶ IU/ml of IL-2, 2-10 μl of 90-110 μg/ml of IL-12, 20-50 μl of 90-110 μg/ml of IL-18, and 4-80 μl of 0.9-1.1 mg/ml of anti-CD16 antibody.

6. The medium composition of claim 5, wherein the additives include, based on 67 ml of the cell culture medium, 650-740 μl of 190-210 mM of L-glutamine and 2-5 ml of autologous plasma.

7. A method for culturing self-activated lymphocytes, the method comprising the steps of:
extracting lymphocytes from human peripheral blood;
culturing the extracted lymphocytes in a medium containing interleukin 2 (IL-2), L-glutamine and autologous plasma in the presence of interleukin 12 (IL-12), interleukin 18 (IL-18), anti-CD3 antibody and anti-CD16 antibody (first culture step); and
adding the lymphocyte-containing medium resulting from the first culture step to a medium containing IL-2, L-glutamine and autologous plasma, followed by culture in the presence of IL-12, anti-CD16 antibody and IL-18 (second culture step).

8. The method of claim 7, wherein the first culture step comprises: culturing the extracted lymphocytes in the medium containing IL-2, L-glutamine and autologous plasma in a first culture container having anti-CD3 antibody solidified therein; transferring the lymphocyte-containing medium in the first culture container into a second culture container, and adding IL-12 and anti-CD16 antibody to the second culture container, followed by culture; and then adding IL-18 to the second culture container, followed by additional culture.

9. The method of claim 7, wherein the first culture step comprises: culturing the extracted lymphocytes in containing IL-2, L-glutamine and autologous plasma for 30 minutes to 2 hours in a first culture container having anti-CD3 antibody solidified therein; transferring the lymphocyte-containing medium in the first culture container into a second culture container, and adding IL-12 and anti-CD16 antibody to the second culture container, followed by culture for 1-3 hours; adding IL-18 to the second culture container, followed by culture for 48-72 hours; and then culturing the lymphocytes for 12-18 hours while adding a medium containing IL-2, L-glutamine and autologous plasma to the second culture container; and then transferring the lymphocyte-containing medium in the second culture container into a third culture container, and adding IL-2, L-glutamine and autologous plasma to the third culture container, followed by additional culture for 2-3 days.

10. The method of claim 7, wherein the second culture step comprises: culturing the lymphocyte-containing medium resulting from the first culture step in a medium containing IL-2, L-glutamine and autologous plasma; and then adding IL-18 to the lymphocyte-containing cells, followed by additional culture.

11. The method of claim 7, wherein the second culture step comprises: adding the lymphocyte-containing medium resulting from the first culture step to a fourth culture container containing a medium supplemented with IL-2, L-glutamine and autologous plasma, followed by culture for 1-3 hours; and then adding IL-18 to the fourth culture container, followed by culture for 12-18 hours; and then adding a medium containing IL-2, L-glutamine and autologous plasma together with anti-CD16 antibody to the fourth culture container and culturing the lymphocytes for 12-18 hours.

12. The method of claim 7, wherein the medium that is used in the first and second culture steps contains 800-1,200 IU (International Unit)/ml of IL-2.

13. The method of claim 7, wherein the method further comprises a step of adding autologous plasma to the lymphocyte-containing medium resulting from the second culture step, and injecting the autologous plasma-containing medium into a gas permeable culture bag containing a medium, followed by culture (third culture step).

14. The method of claim 13, wherein the culture bag that is used in the third culture step contains 100-200 IU/ml of IL-2.
